(19) United States
(12) Patent Application Publication
(10) Pub. No.: US 2005/0244861 A1
(43) Pub. Date: Nov. 3, 2005

(54) Nucleic acids essential for expression of hyphal-specific genes and methods for using the same

(76) Inventor: Paula Sundstrom, Lebanon, NH (US)

Correspondence Address: Licata & Tyrrell P.C., 66 E. Main Street, Marlton, NJ, 08053, US

(21) Appl. No.: 11/082,746

(22) Filed: Mar. 17, 2005

Related U.S. Application Data
(63) Continuation-in-part of application No. 09/725,010, filed on Nov. 29, 2000, now abandoned.
(60) Provisional application No. 60/167,672, filed on Nov. 29, 1999.

(51) Int. Cl. C12Q 1/68; C07H 21/04; C12N 1/18; C12N 15/74

(52) U.S. Cl. 435/6; 435/254.22; 435/483; 536/23.2

(57) ABSTRACT

The present invention relates to the identification of nucleic acids required for the regulation of HWP1 expression in Candida albicans and the use of these nucleic acids in identifying agents which inhibit the expression of HWP1. Such agents can be used in the prevention or treatment of Candida infection of mammalian hosts such as immunocompromised or immunosuppressed humans, for example, those having AIDS or undergoing transplantation or anti-cancer therapy.

### INTRODUCTION

This application is a Continuation-in-Part Application of U.S. patent application Ser. No. 09/725,010, filed Nov. 29, 2000, which claims the benefit of priority from U.S. provisional application Ser. No. 60/167,672, filed Nov. 29, 1999, whose contents are incorporated herein by reference in their entireties.

### BACKGROUND OF THE INVENTION

Fungi are not only important human and animal pathogens, but they are also among the most common causes of plant disease. Fungal infections (mycoses) are becoming a major concern for a number of reasons, including the limited number of antifungal agents available, the increasing incidence of species resistant to known antifungal agents, and the growing population of immunocompromised patients at risk for opportunistic fungal infections, such as organ transplant patients, cancer patients undergoing chemotherapy, burn patients, AIDS patients, or patients with diabetic ketoacidosis. The most common clinical isolate is Candida albicans (comprising about 19% of all isolates). In one study, nearly 40% of all deaths from hospital-acquired infections were due to fungi (Sternberg (1994) Science 266(5191):1632-34.

The yeast Candida albicans (C. albicans) is one of the most pervasive fungal pathogens in humans. It has the capacity to opportunistically infect a diverse spectrum of compromised hosts, and to invade many diverse tissues in the human body. It can in many instances evade antibiotic treatment and the immune system. Although C. albicans is a member of the normal flora of the mucous membranes in the respiratory, gastrointestinal, and female genital tracts, in such locations it may gain dominance and be associated with pathologic conditions. Sometimes it produces progressive systemic disease in debilitated or immunosuppressed patients, particularly if cell-mediated immunity is impaired. Sepsis may occur in patients with compromised cellular immunity, e.g., those undergoing cancer chemotherapy or those with lymphoma, AIDS, or other conditions. Candida may produce bloodstream invasion, thrombophlebitis, endocarditis, or infection of the eyes and virtually any organ or tissue when introduced intravenously, e.g., via tubing, needles, narcotics abuse, etc.

C. albicans has been shown to be diploid with balanced lethals, and therefore probably does not go through a sexual phase or meiotic cycle. This yeast appears to be able to spontaneously and reversibly switch at high frequency between at least seven general phenotypes. Switching has been shown to occur not only in standard laboratory strains, but also in strains isolated from the mouths of healthy individuals.

Oropharyngeal and esophageal candidiasis are among the most frequent opportunistic fungal infections observed in human immunodeficiency virus positive (HIV+) and AIDS patients, occurring in the majority of patients. The pathogenesis is complex and is thought to involve multiple host factors that include loss of cell mediated immunity and altered phagocytic cell activity. The current status of the AIDS epidemic is one of increasing numbers of individuals infected and no cure. Many infected individuals may live for a long time with HIV in an essentially permanent immunocompromised state. Because of the loss of the cellular component of the immune system, AIDS patients are susceptible to invasion of submucosal tissue by C. albicans. The frequency of candidal infections may also be a result of the prophylactic use of antibacterial drugs used in AIDS patients to minimize other opportunistic infections. Candidal infections increase in severity and recur more frequently as the immunodeficiency progresses. Although C. albicans is sensitive to antifungal drugs, treatment over long periods of time are required, and isolates from HIV infected patients may be more resistant that other isolates.

In addition to HIV affected patients, oral candidiasis occurs in cancer patients, e.g., leukemia patients, as well as in patients with other underlying diseases. The most commonly associated disease with oral candidiasis, however, is denture stomatites, which is generally observed in denture wearers.

A feature of C. albicans growth that is correlated with pathogenicity in the oral cavity is the ability to transform from budding to filament-extending growth. Filamentous forms adhere more readily to buccal epithelial cells (BECs) than budding yeasts, and histologically are a prominent feature of invasion of the mucosa. Alternatively, in mucosal and systemic disease, C. albicans exists as a polymorphic set of growth forms termed yeasts, pseudohyphae and true hyphae. In mucosal disease, filamentous forms, particularly true hyphae, invade the keratinized layer of differentiated, stratified squamous epithelium. True hyphae are septate, cylindrical structures with parallel sides that are formed by extension of germ tubes which emerge from yeasts in appropriate environmental conditions. Therefore, knowledge of the molecular events that transform C. albicans to the pathogenic filamentous form as well as detailed investigations of the hyphal surface at the molecular level are necessary for understanding the pathogenesis of candidiasis.

If left untreated, Candida infections frequently lead to the death of the patients. Nystatin, ketoconazole, and amphotericin B are drugs which are used to treat oral and systemic Candida infections. However, orally administered nystatin is limited to treatment within the gut and is not applicable to systemic treatment. Some systemic infections are susceptible to treatment with ketoconazole or amphotericin B, but these drugs may not be effective in such treatment unless combined with additional drugs. Amphotericin B has a relatively narrow therapeutic index and numerous undesirable side effects and toxicities occur even at therapeutic concentrations. While ketoconazole and other azole antifungals exhibit significantly lower toxicity, their mechanism of action, inactivation of cytochrome P₄₅₀ prosthetic group in certain enzymes (some of which are found in humans) precludes use in patients that are simultaneously receiving other drugs that are metabolized by the body's cytochrome P₄₅₀ enzymes. See, e.g., U.S. Pat. No. 5,863,762.

Resistance of bacteria and other pathogenic organisms to antimicrobial agents is an increasingly troublesome problem. The accelerating development of antibiotic-resistant bacteria, intensified by the widespread use of antibiotics in farm animals and overprescription of antibiotics by physicians, has been accompanied by declining research into new antibiotics with different modes of action (Travis (1994) Science 264(5157):360-374). Accordingly, there is a need for an effective treatment of opportunistic infections caused by C. albicans for the aforementioned reasons.

Whether pathogenic or opportunistic, microorganisms have evolved numerous mechanisms to facilitate their establishment and proliferation in mammalian hosts. For example, during initial infection, the interaction between the microorganism and the host may include attachment or adhesion of the microorganism to the host cell surface, invasion of the host cells by the microorganism, and an elaboration of toxins by the microorganism. In certain instances, the microorganism-host cell interaction may be specific or non-specific. Typically, a specific microorganism-host cell interaction may involve the specific binding of the microorganism to a specific receptor or receptor complex expressed on the host cell surface. Subsequently, the binding event may trigger changes in the microorganism and/or the host cell, leading to the progression of infection.

The role of host cell molecules involved in certain microbial interactions has been determined in some cases. Mammalian transglutaminases are examples for which the molecular mechanisms of action and/or the role in host cell growth and development have been elucidated. Generally, transglutaminases are enzymes that catalyze intermolecular crosslinks by the formation of highly stable isopeptide bonds between the γ-carbonyl group of glutamine and the ε-amino group of lysine residues, which are resistance to proteases, sodium dodecyl sulfate, and heat. Epithelial cell transglutaminases are important for the formation of the cornified envelopes of mature squamous epitheilial cells.

It has been shown that certain microorganisms may express proteins capable of acting as substrates for mammalian transglutaminases. One example is the hypha-specific hyphal wall protein 1 (HWP1) of C. albicans. HWP1 is a developmentally-regulated cell wall protein that is expressed exclusively in true hypha in cultures and in animal tissues (Staab, et al. (1999) Science 283(5407):1535-38; Staab, et al. (1996) J. Biol. Chem. 271(11):6298-305; Staab & Sundstrom (1998) Yeast 14(7):681-86).

HWP1 consists of an N-terminal proline and glutamine-rich repetitive amino acid sequence, which is exposed on the hyphal surface, and a cell wall-anchored serine and threonine rich C-terminus. The composition of the N-terminal amino acid repeat is reminiscent of mammalian transglutaminase substrates. HWP1 has a profound effect on adherence of C. albicans to BECs through a novel mechanism that involves host enzymes. HWP1 may serve as a substrate in transglutaminase-mediated cross-linking reactions, thereby forming isopeptide bonds to proteins on the surfaces of host cells. Furthermore, HWP1 encodes an unconventional adhesin that is necessary for the pathogenesis of candidiasis.

### SUMMARY OF THE INVENTION

The present invention is an isolated nucleic acid encoding a Candida albicans HWP1 flanking region, wherein the nucleic acid can be a nucleotide sequence of SEQ ID NO:1 or SEQ ID NO:2; a nucleic acid that hybridizes to a nucleic acid having a nucleotide sequence of SEQ ID NO:1 or SEQ ID NO:2 or its complementary nucleotide sequence under stringent conditions; or a fragment thereof. Vectors and host cells containing an isolated nucleic acid of the invention are also provided.

The present invention is also a method for identifying an agent which modulates the expression of HWP1. The method involves contacting a test agent with a host cell expressing a reporter protein, wherein the nucleic acid encoding the reporter is operably linked to an HWP1 nucleic acid of the present invention, and determining whether the test agent modulates the expression of the reporter protein thereby identifying an agent which modulates the expression of HWP1.

### BRIEF DESCRIPTION OF THE DRAWINGS

FIG. 1 depicts the sequence of the 5′ flanking region of HWP1. Part of the upstream region of HWP1 containing regulatory elements was isolated as a 1.6 BglII fragment of C. albicans genomic DNA. The cloned HWP1 promoter region includes 1,467 kb upstream of the ATG start codon of the structural HWP1 gene. The promoter additionally includes a ˜600 bp region located downstream of the APL6 locus. A putative TATA box was found 67 bp upstream from the beginning of the transcript.

FIG. 2 illustrates the comparison between the GAT99 50-residue DNA binding domain (SEQ. ID. NO: 23) with the NIT2 50-residue DNA binding domain (SEQ. ID. NO: 24). A search of the C. albicans genome revealed that GAT99 is highly homologous to NIT2. Using the ALIGN program, 92% homology was observed between the two DNA binding domains. The asterisks (*) indicate cysteine residues involved in zinc chelation, and the diamond identifies the leucine residue, which when altered in AREA and NIT2 lead to different changes in promoter recognition.

### DETAILED DESCRIPTION OF THE INVENTION

A hallmark of tissue invasion by the yeast C. albicans is the preponderance of hyphal forms during growth within tissue. This observation prompts the hypothesis that the ability to form hyphae is responsible for the pathogenesis of candidiasis. However, several lines of evidence suggest that it is not necessarily growth as hyphae that is essential for disease, but rather it is the expression of pro-invasive factors that coincide with hyphal growth that mediates tissue invasion. In some cases, the lack of filament production is correlated with a loss in virulence (Csank, et al. (1998) Infect. Immun. 66(6):2713-21; Ghannoum, et al. (1995) Infect. Immun. 63(11):4528-30; Lo, et al. (1997) Cell 90(5):939-49; Zaragoza, et al. (1998) J. Bacteriol. 180(15):3809-15) but other examples exist where deletion of a specific gene product does not impair hyphal growth in tissues or in laboratory cultures, yet virulence is reduced in animal models (De Bernardis, et al. (1999) J. Infect. Dis. 179(1):201-8; Leidich, et al. (1998) J. Biol. Chem. 273(40):26078-86). Moreover, a mutant that grows obligately in filamentous forms does not show enhanced virulence (Braun & Johnson (1997) Science 277(5322):105-09; De Bernardis, et al. (1999) supra; Leidich, et al. (1998) supra). Although some studies have suggested that mutant strains lacking the ability to form hyphae are avirulent, it is not known whether the loss of virulence is a result of the lack or hyphae per se, or a lack of specific pro-invasive factors that are expressed during hyphal growth. Finally, studies have shown that strains of C. albicans isolated from human patients that are uniformly successful in forming hyphae, vary in their ability to invade tissue in a rat tongue model (Allen, et al. (1990) J. Oral Pathol. Med. 18(6):352-59). These observations all point to the presence of pro-invasive factors that if present in a strain, are produced during hyphal growth. Thus, hyphal growth may be necessary but not sufficient for candidiasis to occur. In order to understand the pathogenesis of candidiasis, it is important to identify proteins that contribute to pathogenesis, and then to discover factors that regulate their expression. The ability to interfere with mechanisms of expression of pro-invasive genes will lead to the development of strategies to interfere with candidiasis.

Hwp1 is a developmentally-regulated cell wall protein that is expressed exclusively in true hyphae in cultures and in animal tissues. Hwp1 has a profound effect on adherence to human BECs through a novel mechanism that involves host enzymes, and is important for systemic candidiasis in mice. HWP1 expression is tightly regulated at the level of mRNA; cultures growing exclusively as yeasts lack HWP1 mRNA, whereas in cultures of true hyphae, HWP1 mRNA is abundant, e.g., even more abundant than genes for glycolytic enzymes. Thus, developmental expression is regulated by differences in transcription of HWP1 in yeast and hyphal growth forms. Flanking regions of the HWP1 structural gene contain regulatory sequences that affect the levels of HWP1 mRNA. A corollary is that HWP1 is a target of a gene regulatory circuit that also controls expression of other hypha-specific genes that are pathogenically important.

Accordingly, the present invention is an isolated nucleic acid corresponding to a region of DNA flanking the coding sequence of HWP1 and the use thereof in identifying agents which regulate the expression of HWP1 and therefore adhesion of C. albicans to host cells. As used herein, an isolated nucleic acid means a molecule separated or substantially free from at least some of the other components of the naturally occurring organism, such as for example, the cell structural components or other polypeptides or nucleic acids commonly found associated with the molecule.

The term nucleic acid sequence includes an oligonucleotide, nucleotide, or polynucleotide, and fragments thereof, and to DNA or RNA of genomic or synthetic origin which can be single- or double-stranded, and represent the sense or antisense strand, peptide nucleic acid (PNA), or any DNA-like or RNA-like material, natural or synthetic in origin.

One embodiment of the instant invention is an isolated upstream region of the Candida HWP1 locus, wherein the upstream region is set forth herein as SEQ ID NO:1. The upstream region as disclosed herein, includes the HWP1 promoter the 5′ untranslated region of the HWP1 transcript. A promoter is generally referred to as a regulatory region of DNA located upstream or 5′ of a coding sequence which is capable of initiating, directing and mediating the transcription of a nucleic acid sequence. Promoters can additionally comprise recognition sequences, such as upstream or downstream promoter elements, which can influence the transcription rate.

Another embodiment of the present invention is an isolated 3′ flanking region of the Candida HWP1 locus, wherein the 3′ flanking region is set forth herein as SEQ ID NO:2. The 3′ flanking region as disclosed herein, includes the 3′ untranslated region of the HWP1 transcript and sequences located downstream thereof. In general, 3′ flanking regions provide the appropriate signals for termination of transcription and mRNA stability and, in some cases, may contain enhancer or repressor sequences.

In some embodiments, a nucleic acid of the invention is a fragment of a nucleic acid of SEQ ID NO:1 or SEQ ID NO:2. A fragment of a nucleic acid of SEQ ID NO:1 or SEQ ID NO:2 can be a truncated portion of SEQ ID NO:1 or SEQ ID NO:2 (i.e., missing nucleotides from the 5′ or 3′ ends) which retains all or a part (e.g., 50%, 60%, 70%, 80%, 90% or more) of the regulatory capacity of the full-length sequence. A fragment can also be a 10, 20, 30, 40, 50, 60, 100, or up to 500 bp portion of SEQ ID NO:1 or SEQ ID NO:2 which retains at least a part of the regulatory capacity of the full-length sequence. Exemplary fragments of the HWP1 promoter include upstream regulatory elements located at −1902 to −1410 (designated MRR1 herein; SEQ ID NO:3) and −1410 to −1042 (designated MRR2 herein; SEQ ID NO:4) of the HWP1 promoter (i.e., upstream of the HWP1 transcription start site). MRR2 has been found to contain a nucleotide sequence essential for activation of the HWP1 promoter, whereas MRR1 amplifies the effect of MRR2. Other suitable fragments of the HWP1 promoter include sequences represented by SEQ ID NO:8-22.

In still other embodiments, a nucleic acid of the invention is a nucleic acid that hybridizes to a nucleic acid having a sequence of SEQ ID NO:1 or SEQ ID NO:2 or its complementary nucleotide sequence under stringent conditions. To illustrate, hybridization of such sequences can be carried out under conditions of reduced stringency, medium stringency or even stringent conditions (e.g., conditions represented by a wash stringency of 35-40% Formamide with 5× Denhardt's solution, 0.5% SDS and 1×SSPE at 37° C.; conditions represented by a wash stringency of 40-45% Formamide with 5× Denhardt's solution, 0.5% SDS, and 1×SSPE at 42° C.; and/or conditions represented by a wash stringency of 50% Formamide with 5× Denhardt's solution, 0.5% SDS and 1×SSPE at 42° C., respectively) to the sequences specifically disclosed herein. See, e.g., Sambrook et al., Molecular Cloning, A Laboratory Manual (2d Ed. 1989) (Cold Spring Harbor Laboratory).

It will be appreciated by those skilled in the art that there can be variability in the nucleic acid sequence of HWP1 5′ and 3′ sequences of the present invention due to Candida strain and species variability. Accordingly, the isolated nucleic acids of the invention encompass those nucleic acids having at least about 60%, 70%, 80%, 90%, 95%, 97%, 98% or higher amino acid sequence identity with the sequences specifically disclosed herein (or fragments thereof). In particular embodiments, an isolated nucleic acid of the present invention shares at least 90%-99% identity with SEQ ID NO:1 or SEQ ID NO:2. In other embodiments, an isolated nucleic acid of the present invention shares at least 95%-99% identity with SEQ ID NO:1 or SEQ ID NO:2.

As is known in the art, a number of different programs can be used to identify whether a nucleic acid has sequence identity or similarity to a known sequence. Sequence identity can be determined using standard techniques known in the art, including, but not limited to, the local sequence identity algorithm of Smith & Waterman (1981) Adv. Appl. Math. 2:482, by the sequence identity alignment algorithm of Needleman & Wunsch (1970) J. Mol. Biol. 48:443, by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux, et al. (1984) Nucl. Acid Res. 12:387-395, either using the default settings, or by inspection.

An example of a useful algorithm is the BLAST algorithm, described in Altschul, et al. (1990) J. Mol. Biol. 215:403-410 and Karlin, et al. (1993) Proc. Natl. Acad. Sci. USA 90:5873-5787. A particularly useful BLAST program is the WU-BLAST-2 program which was obtained from Altschul, et al. (1996) Methods in Enzymology, 266:460-480; http://blast.wustl/edu/blast/README.html. WU-BLAST-2 uses several search parameters, which can be set to the default values. The parameters are dynamic values and are established by the program itself depending upon the composition of the particular sequence and composition of the particular database against which the sequence of interest is being searched; however, the values can be adjusted to increase sensitivity. An additional useful algorithm is gapped BLAST as reported by Altschul, et al. (1997) Nucleic Acids Res. 25:3389-3402.

A percentage nucleic acid sequence identity value can be determined by the number of matching identical nucleotides divided by the total number of nucleotides of the longer sequence in the aligned region. The longer sequence is the one having the most actual residues in the aligned region (gaps introduced by WU-Blast-2 to maximize the alignment score are ignored).

The alignment can include the introduction of gaps in the sequences to be aligned. In addition, for sequences which contain either more or fewer nucleotides than the nucleic acids specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence. In percent identity calculations relative weight is not assigned to various manifestations of sequence variation, such as, insertions, deletions, substitutions, etc.

A nucleic acid of the present invention can be included in any one of a variety of vectors, in particular vectors or plasmids for expressing a polypeptide of interest. A typical expression vector can contain a promoter, selection marker, nucleic acids encoding signal sequences, and the like. Vectors include viral derived vectors, bacterial derived vectors, plant derived vectors and insect derived vectors. Such vectors include chromosomal, nonchromosomal and synthetic DNA sequences, e.g., derivatives of SV40; bacterial plasmids; phage DNA; yeast plasmids; vectors derived from combinations of plasmids and phage DNA; viral DNA such as vaccinia, adenovirus, fowl pox virus, and pseudorabies. However, any other vector or plasmid can be used as long as they are replicable and viable in the host.

The appropriate nucleic acid can be inserted into the vector by a variety of procedures. In general, the nucleic acid is inserted into an appropriate restriction endonuclease sites by procedures known in the art and subsequently transformed into a cultured host cell for vector maintenance or recombinant protein expression. Such procedures and others are deemed to be within the scope of those skilled in the art.

Having identified several key sequences (e.g., MRR1 and MRR2) in the HWP1 promoter, it will be appreciated by the skilled artisan that other regulatory elements and regulatory proteins which bind to these regions can now be readily identified. Further, the regulatory elements and regulatory proteins thereof can be employed in screening methods for identifying agents which inhibit expression of HWP1 and therefore adhesion of C. albicans.

The regulation of expression of hypha-specific genes is controlled by the presence of mRNA in hyphae but not in yeasts. The presence of multiple genes that are correlated with hyphal growth, and the common control determined by the presence of mRNA, indicates that common regulatory mechanisms exist for inducing expression of hypha-specific genes. Initiation of transcription control is mediated by cis-acting sequences, termed gene response elements, that interact with DNA binding proteins to repress or activate transcription in response to environmental signals. Gene response elements serve to activate or repress gene expression though interactions with DNA binding proteins. Exemplary hypha-specific gene response elements include MRR1 and MRR2. It is contemplated that MRR1 and MRR2 elements could be common cis-acting elements which function in the coordinate regulation of a set of genes that are appropriate for a given set of environmental conditions.

Other regulatory elements which may play a role in the regulation of C. albicans HWP1 expression under various environmental conditions include, but are not limited to, NIT2, PHO4, and upstream regulatory sequence (URS) elements such as those for TUP1 and NRG1. C. albicans strains lacking TUP1 or NRG1 grow exclusively in filamentous form (Braun & Johnson (1997) Science 277(5322):105-09; Murad, et al. (2001) EMBO J. 20:4742-52) and HWP1 was found to be deprepressed in both tup1 and nrg1 mutants (Sharkley, et. al. (1999) supra; Murad, et al. (2001) supra). However, when tup1 mutants were analyzed, surface HWP1 protein was not detected on hyphal surfaces under most culture conditions, suggesting that release from TUP1 repression permits only partial HWP1 expression and that additional proteins are required for normal levels and surface localization of hypha-specific gene products. The partial derepression of HWP1 in tup1 mutant indicates that proteins other than TUP1 and NRG1 repress HWP1 in yeasts. Regulatory elements for the C. albicans transcription factors NRG1, RFG1 and EFG1 may play a direct role in the regulation of HWP1.

Additional regulatory elements can be identified in accordance with the methods employed in the examples disclosed herein or any other art-established method. For example, upstream activating sequence (UAS) elements can be identified by designing individual, overlapping bp sequence elements spanning the entire DNA sequence, and then looking for activation of reporter gene expression relative to a simple promoter containing a TATA element (Guarente & Ptashne (1981) Proc. Natl. Acad. Sci. USA 78(4):2199-203; Rupp, et al. (1999) EMBO J. 18(5):1257-69).

As indicated supra, upstream regulatory elements of HWP1 (e.g., full-length promoter, MRR1 or MRR2) can be used in a screening method for identifying agents that inhibit or decrease the expression of a product of nucleic acids encoding HWP1 by at least around 25%, 50% 75%, 98%, or 100% relative to a control. Such a method involves contacting a test cell (e.g., C. albicans), which contains nucleic acids encoding a reporter operably linked to an HWP1 regulatory element, with a test agent; incubating the plate under appropriate environmental conditions (e.g., hyphae inducing conditions) to induce HWP1 expression for a time sufficient to allow the test agent to effect GFP accumulation; detecting fluorescence of the test cells contacted with the test agent, wherein fluorescence indicates expression of the GFP polypeptide in the test cells; and comparing the fluorescence of test cells contacted with the test agent to cells not contacted with the test agent. A decrease in fluorescence of the test cell contacted with the test agent relative to the fluorescence of test cells not contacted with the test agent indicates that the test agent causes a decrease in expression of products of nucleic acids encoding HWP1 in the test cell.

As used herein, operably linked is intended to mean that nucleic acids encoding the reporter are functionally linked to the HWP1 regulatory element such that the HWP1 regulatory element mediates transcriptional regulation of the reporter nucleic acids into mRNA and translation of the mRNA into the protein.

While, beta-glucuronidase, and luciferase can be employed in accordance with the instant method, green fluorescent protein (GFP) has proven to be a valuable reporter of gene expression in studies employing optical microscopy in research in microbial pathogenesis. The advantages of GFP have been made available for studies in C. albicans by optimization of all the GFP codons for expression in C. albicans (Cormack, et al. (1997) Microbiol. 143(Pt 2):303-11). GFP within C. albicans is readily visible by fluorescence microscopy and is easily quantified by flow cytometry. GFP in solution is also quantifiable by fluorometry (Kahn, et al. (1997) Curr. Biol. 7(4):R207-08). These advances indicate that GFP is a suitable reporter for studies on inducible expression of HWP1 in C. albicans.

Test agents which can be screened in accordance with the screening assay provided herein encompass numerous chemical classes, though typically they are organic molecules, preferably small organic compounds having a molecular weight of more than 100 and less than about 2,500 daltons. Libraries of such compounds can contain either collections of pure agents or collections of agent mixtures. Examples of pure agents include, but are not limited to, proteins, antibodies, peptides, peptide aptamers, nucleic acids, oligonucleotides, carbohydrates, lipids, synthetic or semi-synthetic chemicals, and purified natural products. Such libraries are commercially available to the skilled artisan. Examples of agent mixtures include, but are not limited to, extracts of prokaryotic or eukaryotic cells and tissues, as well as fermentation broths and cell or tissue culture supernates. In the case of agent mixtures, the methods of this invention are not only used to identify those crude mixtures that possess the desired activity, but also provide the means to monitor purification of the active agent from the mixture for characterization and development as a therapeutic drug. In particular, the mixture so identified can be sequentially fractionated by methods commonly known to those skilled in the art which can include, but are not limited to, precipitation, centrifugation, filtration, ultrafiltration, selective digestion, extraction, chromatography, electrophoresis or complex formation. Each resulting subfraction can be assayed for the desired activity using the original assay until a pure, biologically active agent is obtained.

Library screening can be performed in any format that allows rapid preparation and processing of multiple reactions. Stock solutions of the test agents as well as assay components are prepared manually and all subsequent pipetting, diluting, mixing, washing, incubating, sample readout and data collecting is done using commercially available robotic pipetting equipment, automated work stations, and analytical instruments for detecting the signal generated by the assay, i.e., reporter protein activity.

Furthermore, the invention provides agents identified as inhibitors of expression of products of nucleic acids encoding HWP1 and methods for using these agents to decrease, repress, or inhibit adhesion of C. albicans, thereby preventing or reducing candidiasis in a subject. These agents can be incorporated into a pharmaceutical composition and administered to a subject (e.g., a human) at risk or in need of such treatment.

Pharmaceutical compositions of the present invention contain an effective amount of an agent which alters the expression of a product of a nucleic acid encoding HWP1 and a pharmaceutically acceptable vehicle. By effective amount it is meant an amount which inhibits or decreases the expression of a product of a nucleic acid encoding HWP1 and renders C. albicans unable to adhere to host cells. Such pharmaceutical compositions can be prepared by methods and contain vehicles which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. For example, sterile saline and phosphate-buffered saline at physiological pH can be used. Preservatives, stabilizers, dyes and even flavoring agents can be provided in the pharmaceutical composition. For example, sodium benzoate, sorbic acid and esters of p-hydroxybenzoic acid may be added as preservatives. In addition, antioxidants and suspending agents can be used.

DNA binding proteins, which bind to and regulate the expression of HWP1, can also be used in screening assays to identify agents which regulate the expression of HWP1. Such agents would function by binding to and interfering with the ability of the DNA binding protein to bind to the HWP1 regulatory element thereby decreasing expression of HWP1. Evidence for the presence of unknown DNA binding proteins is provided by electrophoretic mobility shift assays, where proteins in crude nuclear extracts of cells growing in appropriate conditions are assayed for their ability to retard the migration of labeled regulatory elements in polyacrylamide gels. Mutations or deletions in the regulatory elements are employed to verify the specificity of the interactions of DNA binding proteins with the regulatory element and to map the most important nucleotides for DNA binding. The presence of unknown binding proteins to regulatory elements provides the groundwork for cloning genes encoding regulatory proteins.

Several strategies have been successfully used to clone genes encoding proteins that bind to regulatory elements. A particularly successful method is to use DNA affinity beads that consist of regulatory elements that have incorporated biotinylated nucleotides to facilitate coupling to streptavidin beads (Gabrielsen & Huet (1993) Methods Enzymol. 218:508-25). Nuclear extracts are then incubated with the beads to separate the desired DNA binding proteins from other proteins in the extract, followed by elution of the DNA binding protein from the beads, purification on SDS polyacrylamide gels and amino acid sequencing of HPLC-purified proteolytic fragments. The amino acid sequences are then used to design degenerate primers for amplification of a PCR product from cDNA to be used as a probe in library screening to obtain the DNA binding protein gene. Verification of the biological activity of the DNA binding protein is gained from creating null mutants in the gene and looking for loss of expression of the reporter gene under the control of the specific regulatory elements used to identify and characterize the protein. The pattern of expression of the DNA binding protein gene itself, along with the predicted primary amino acid sequence, provide insight into regulatory mechanisms. Predictions about whether expression is mediated by transcription, or by protein modification, are possible. In the event that the DNA binding protein gene is regulated by transcription, its non-flanking regions provide material for identification of cis-acting regulatory regions for the primary target gene, i.e., HWP1.

One of the benefits of cloning gene(s) encoding proteins that bind to the promoter of a target gene like HWP1 is that global gene regulatory networks can be readily sought. Target genes that are believed to be activated by the same mechanism will have reduced levels of mRNA in a null mutant lacking the DNA binding protein gene. For example, expression of sporulation-specific genes of S. cerevisiae is regulated by the IME1 gene. Deletion of IME1 results in the loss of expression of an entire family of sporulation-specific genes (Mitchell (1994) Microbiol. Rev. 58(1):56-70). In accordance with the present invention, known hypha-specific genes are assessed for expression in a null mutant of the DNA binding protein gene compared to cells having the gene, thereby revealing the importance of the DNA binding protein in regulating a set of genes.

Opportunities for proliferation and invasion of mammalian hosts by C. albicans are continuing to increase. Because of the loss of the cellular component of the immune system, AIDS patients are susceptible to invasion of submucosal tissue by C. albicans. The frequency of candidal infections may also be a result of the prophylactic use of antibacterial drugs used in AIDS patients to minimize other opportunistic infections. Candidal infections increase in severity and recur more frequently as the immunodeficiency progresses. In non-AIDS patients, such as those undergoing organ transplantation, are neutropenic, or have debilitating diseases requiring advanced modalities of life support, mucosal and hematogenously disseminated candidiasis seriously threaten optimal treatment outcomes. While antifungal drugs can be effective, the increasing frequency of resistant strains of C. albicans, and the systemic side effects of the drugs prompts exploration of novel strategies to interrupt the sequence of events leading to disease and to expand the repertoire of antifungal drugs. Both goals require improved understanding of hypha-specific proteins of C. albicans in terms of their roles in pathogenicity and in the mechanisms that regulate their expression. By defining the molecular events leading to the expression of a pathogenically important adhesin, and through the identification of new genes that are co-regulated with HWP1 in a putative global regulatory circuit, the present invention relates to new and novel ways to interfere with candidiasis. The long-term medical benefits is the development of alternative or adjunctive therapies based on the knowledge expression of hypha-specific genes in C. albicans.

The invention is described in greater detail by the following non-limiting examples.

### EXAMPLE 1

### Structural Features of the Gene Product of HWP1

The complete HWP1 gene was obtained by screening a C. albicans genomic library with cDNA which had been isolated following immunoscreening of a germ tube cDNA library with hypha-specific antibodies (Staab, et al. (1996) supra).

HWP1 encodes a protein of 634 amino acids with an N-terminal antigenic domain that is more hydrophilic, more acidic, more likely to be present on the protein surface and has a higher antigenic index than the remainder of HWP1. In contrast to the uniqueness of the antigenic domain that was previously described, the central and C-terminal regions of HWP1 shares features that have been found in other yeast cell wall proteins, including a high percentage of serine and threonine residues that leads to extensive O-linked carbohydrate modification. The two serine/threonine-rich regions (from Ile₁₈₈ to Cys₃₈₉, and from Thr₃₉₀ to Glu₆₁₂) are distinguishable by their mole percent of these two amino acids. In the first and second regions respectively, 54% and 30% of the residues are serine or threonine. The different content of serine and threonine residues may reflect distinct functions for each region. The second region is also rich in alanine and glutamine (20% of residues) and has a higher content of proline residues than the first region. The discovery of an abrupt increase in serine and threonine residues at the C-terminal boundary of the antigenic domain of HWP1 indicates that the “lollipop on a stick” model, in which a C-terminal domain rich in hydroxy amino acids serves to extend a binding domain above the cell surface, may explain the exposure of the antigenic domain of HWP1 at the cell surface (Jentoft (1990) Trends Biochem. Sci. 15:291-94). The absence of glycosylation sites resulting in a lack of carbohydrate masking in the N-terminal repetitive domain indicates that this peptide sequence is important for interactions with host surfaces.

The first serine/threonine region shows similarity (BLASTP algorithm; Altschul, et al. (1990) J. Mol. Biol. 215:403-10) to mucins from different species because of small stretches of threonine residues in HWP1, while the second serine/threonine region did not show similarity to proteins in the databanks. Two, 29 amino acid direct repeats separated by 5 residues were found in this latter region. The amino acid repeats were identical except for a conserved change of alanine to valine at position 22 within the repeat. In addition to multiple sites for O-linked carbohydrate addition, three potential N-glycosylation sites were identified at amino acid positions 241, 286 and 601, all of which were Asn-Xaa-Ser. Further, the hydrophobic C-terminus in HWP1 was similar to other yeast cell membrane or cell wall proteins whose maturation involves transfer to a glycosylphosphatidylinositol (GPI) anchor.

HWP1 is acidic, having 63 negative charges at neutral pH, and an isoelectric point of 3.37, which is expected to contribute to the strong negative charge of surfaces of C. albicans hyphae. Following signal peptidase cleavage after amino acid 39, HWP1 has a calculated Mr of 61,122 (Staab, et al. (1996) supra).

The hydrophobic N- and C-termini are consistent with entry into the classical secretory pathway and modification with a GPI anchor (Englund (1993) Annu. Rev. Biochem. 62:121-38) respectively, and the C-terminal 26 amino acids of Hwp1 have been shown to be essential for presentation of Hwp1 on the surface of germ tubes (Staab, eta l. (2004) J. Biol. Chem. 279:40737-40.

### EXAMPLE 2

### Sequence of the 5′Flanking Region of HWP1

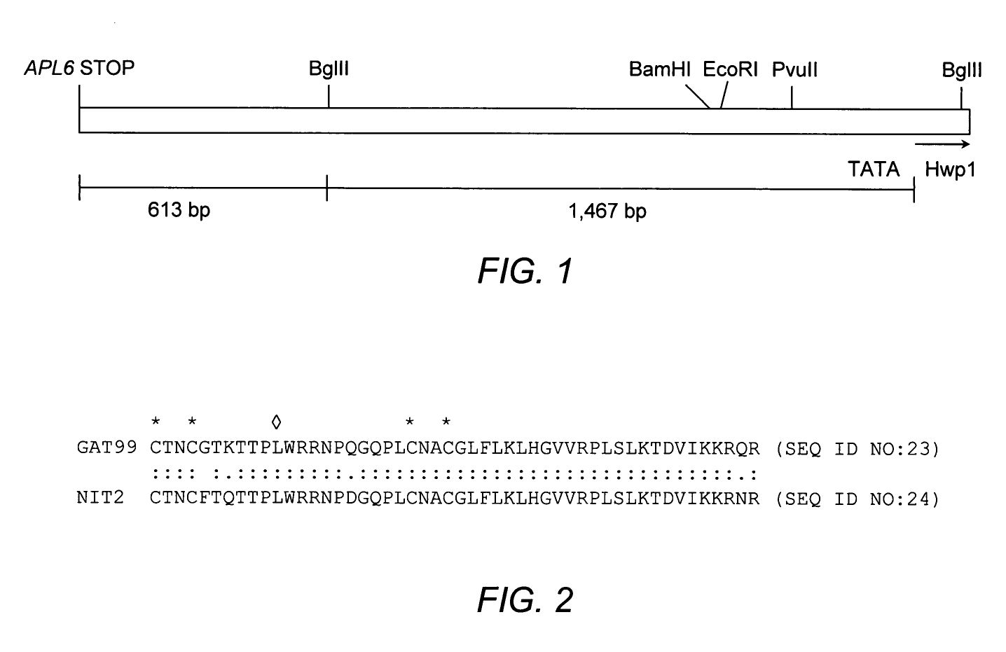

Part of the upstream region of HWP1, containing the promoter and regulatory sequences, was isolated as a 1.6 kbp BglII fragment of C. albicans genomic DNA. The cloned HWP1 promoter region includes 1,467 bp upstream of the ATG start codon of the structural HWP1 gene and ˜600 bp of sequence located 3′ of APL6. Genetic organization of the upstream region of HWP1 is shown in FIG. 1 and set forth as SEQ ID NO:1.

Analysis of the promoter of HWP1 identified a 10 nucleotide perfect direct repeat (CTGAATTATC; SEQ ID NO:5) and a putative TATA box (i.e., TATAAAT; SEQ ID NO:6). Searches for potential binding sites in fungal matrices using MatInspector v2.2 revealed the presence of sequences for NIT2 (16 sites), PHO4 (2 sites), MATA1 (1 site), HSF (1 site), HAP2/3/5 (3 sites), AbaA (1 site), HMG-BOX (7 sites), qa-1F (2 sites), AMT1/ACE1 (1 site), GCN4 (3 sites), M cell-specific TR-box (2 sites), GCR1 (1 site), NGR1 (4 sites) and EFG1 (10 sites) binding sites within the HWP1 promoter. Select binding sites are listed in Table 1. A S. cerevisiae cAMP response element (CRE; GCACGTTA; SEQ ID NO:7) site was also found within a PHO4 site. The presence of NIT2 and HSF sites are of interest as there is evidence for nitrogen regulation and temperature regulation of germ tube formation in C. albicans. A notable feature is a pair of NIT2 sites in opposite orientations near position 800 that form an almost perfect palindrome on opposite DNA strands. The presence of HMG-BOXes may confer repressor regions in light of the suppressive effect of Rfg1, a C. albicans HMG protein, on filamentation (Khalaf and Zitomer (2001) Genetics 157:1503-12; Kadosh and Johnson (2001) Mol. Cell. Biol. 21:2496-505). Of further significance are the putative E-boxes (10 sites) for the C. albicans transcription factor Efg1 and NRE elements for the Nrg1 filamentation repressor (2 sites) (Leng, et al. (2001) J. Bacteriol. 183:4090-3; Murad, et al. (2001) supra).

TABLE 1

| | | | | |
|---|---|---|---|---|
| | | | | SEQ ID |
| Element | Location* | Orientation | Sequence | NO: |

| | | | | |
|---|---|---|---|---|
| NIT2 | 59 | 3′to 5′ | ATGATA | 8 |
| | 228 | 3′to 5′ | CGGATA | 9 |
| | 265 | 3′to 5′ | ACGATA | 10 |
| | 433 | 3′to 5′ | GCGATA | 11 |
| | 560 | 5′to 3′ | TATCCA | 12 |
| | 573 | 5′to 3′ | TATCAG | 13 |
| | 705 | 5′to 3′ | TATCCA | 14 |
| | 800 | 3′to 5′ | ATGATA | 8 |
| | 807 | 5′to 3′ | TATCAA | 15 |
| | 921 | 5′to 3′ | TATCTT | 16 |
| | 1139 | 3′to 5′ | GGGATA | 17 |
| | 1246 | 5′to 3′ | TATCAC | 18 |
| | 1255 | 5′to 3′ | TATCCG | 19 |
| | 1321 | 5′to 3′ | TATCAT | 20 |
| | 1416 | 5′to 3′ | TATCAA | 15 |
| PHO4 | 94 | 5′to 3′ | AAGCACGTTTGA | 21 |
| | 468 | 5′to 3′ | TTGCACGTTAGC | 22 |

*Sequence location is that in SEQ ID NO:1.

The numerous sites for NIT2 binding are significant because the NIT2 protein of Neurospora crassa is a global positive-acting transcription factor of nitrogen structural genes when preferred nitrogen sources are lacking. A NIT2-like protein likely may regulate virulence genes in C. albicans.

A search of the C. albicans genome revealed that a region of GAT99 (SEQ ID NO:23) shares 92% identity with the 50-residue DNA binding domain of NIT2 (SEQ ID NO:24)(FIG. 2). This region is highly conserved in GATA factors. The GAT99 (GAT-1 or GATA like protein) predicted amino acid sequence was also homologous to AREA, NRE and GLN-3. Finding GAT99 in C. albicans suggests that regulation of HWP1 expression could be tied to nitrogen regulatory circuits. In fungi, NIT2 sites in promoter regions vary in number, location, and orientation; strong NIT2 binding sites are usually found as two or more elements located within 30 bp of each other in the same or opposite orientation. There are three such pairs of NIT2 sites in the 1467 bp upstream region of HWP1, one set starting at bp 560, the second at bp 800 and the third at bp 1246.

### EXAMPLE 3

### Sequence of the 3′Flanking Region of HWP1

1,441 nucleotides downstream of HWP1 were cloned and sequenced. The end of transcription was determined by 3′ RACE PCR using an HWP1-specific oligonucleotide corresponding to residues 691-710. The 3′ DNA sequence of the RACE PCR product showed that translation of HWP1 terminated with a UAA codon followed by 277 nucleotides of untranslatead message. A polyadenylation site (AAUAAA; SEQ ID NO:25) 252 nucleotides from the stop codon, and a poly A tail of 14 adenosines were also part of the 3′ region of the message. A truncated open reading frame consisting of 178 amino acids, beginning with an ATG and interrupted by the BamHI site at the end of the cloned 3′ region of HWP1 was identified as a homologue of RAD2 (excision repair gene). Studies of the open reading frame showed that this gene is not differentially regulated (mRNA is present in both yeast and hyphal forms).

### EXAMPLE 4

### Kinetics of Expression of HWP1 mRNA

Initial studies showed the presence of an abundant HWP1 signal on northern blots three hours after initiation of mass conversion from yeasts to hyphae (strain SC5314) in M199 medium. To gain more information about the kinetics of expression of HWP1, northern blot analysis was performed on cells placed in prewarmed M199, Lee's pH 6.5 medium, and M199 with 5% fetal calf serum beginning at 10 minutes and continuing for 7 hours. Under these conditions, close to 100% of the cells form germ tubes; no budding yeasts were observable. Each lane contains 2.5 μg of total RNA. The presence of nearly equivalent amounts of RNA in all lanes was verified by using an 18S ribosomal RNA probe which controls for sample amounts in lanes. Oligonucleotide primers representing nucleotides 314 to 332 and complementary to 967 to 986 of 18S rRNA gene were used to amplify a 672 bp fragment by PCR. The PCR product was radiolabeled and used to probe stripped northern blots. The blots were first probed with HWP1 cDNA which represents the 5′ region of the structural gene that encodes the proline and glutamine-rich repetitive N-terminal domain HWP1.

The blot was overexposed and showed that HWP1 mRNA was detectable within 20 minutes and up to 7 hours of placing cells under induction conditions. The message level increased dramatically between 20 and 30 minutes and continued to rise, peaking at 3-4 hours. After four hours, the HWP1 mRNA began to decline until 7 hours, where it stabilized at a low level.

To correlate the presence of HWP1 mRNA with protein (HWP1) and germ tube formation, indirect immunofluorescence experiments were performed using monospecific antiserum to recombinant HWP1 (rHWP1). Germ tube production and surface HWP1 production were synchronized, both appearing at 45 minutes on approximately 25% of yeasts and were seen on the majority of cells by 60 minutes. Messenger RNA preceded the presence of visible germ tubes and surface HWP1 by 20-25 minutes.

The kinetics of HWP1 expression appeared to differ quantitatively from mRNA for housekeeping genes, but the overall rise and fall of message levels was similar. HWP1 message was evident nearly an hour earlier than ENO mRNA, a gene which encodes an abundant glycolytic enzyme, enolase, that is present in yeast and hyphal growth. However, both mRNA's peaked at 3-4 hours and dropped thereafter. Similar results were found for other non-developmentally regulated genes ACT1 and carboxypeptidase Y. These results show that expression of HWP1 is produced earlier and to higher levels than housekeeping genes, reflecting the presence of activation mechanisms not found for housekeeping genes or at least in addition to those for housekeeping genes.

The results indicate that repressive mechanisms serve to shut off HWP1 during yeast growth, through interactions of HWP1 upstream sequences and DNA-binding proteins that interact with TUP1 or other general repressor proteins. The results also show that activating factors serve to increase HWP1 mRNA levels above those for other abundant proteins such as enolase and actin during germ tube growth.

### EXAMPLE 5

### HWP1:GFP Reporter Fusion

HWP1:GFP reporter fusion was created for monitoring the HWP1 promoter and for verifying that the appearance of GFP accurately reflects the activity of the native HWP1 promoter.

A transcriptional fusion between HWP1 flanking regions and a reporter gene (GFP) was created and targeted to the genomic enolase locus. The reason for inserting the reporter into the genome was to avoid artifacts associated with overexpression and problems with plasmid instability that are inherent in the use of C. albicans ARS (CARS) plasmids. The ENO genomic locus was selected for convenience and because phenotypes associated with disruption at the ENO locus of C. albicans are known; disruption of one of the C. albicans enolase genes does not adversely affect growth, morphogenesis (Postlethwait & Sundstrom (1995) J. Bacteriol. 177:1772-9; Staab, et al. (1999) supra) or pathogenesis in a murine model of systemic candidiasis.

To easily assess the role of HWP1 flanking sequences in gene expression, the coding region of HWP1 was replaced with that of the green fluorescent protein (GFP) which has been optimized for expression in C. albicans (Cormack, et al. (1997) supra). The transformation recipient was ura auxotroph CAI4. The presence of a functional HWP1 gene in this strain was used to correlate the expression of GFP message with HWP1 message, confirming the utility of the reporter strain as accurately reporting HWP1 expression.

The features of the resulting reporter construct, pHWP1GFP3, included low copy number; 1,467 bp of HWP1 upstream sequence (i.e., SEQ ID NO:1) and 352 bp of HWP1 3′ sequence flanking GFP; and the C. albicans enolase gene disrupted with the C. albicans URA3 gene for selection in the ura3 strain CAI4, and for targeting to of one of the ENO gene loci of C. albicans.

### EXAMPLE 6

### GFP Expression

Plasmid HWP1GFP3 was digested with ClaI which cuts at a single site in the C. albicans enolase gene to target the construct to the ENO locus in the genome using spheroplast transformation. This resulted in a mutated copy of the enolase gene disrupted with URA3 adjacent to a wild-type copy. Transformants were selected on media lacking uridine. A control plasmid lacking HWP1 upstream regions but otherwise identical to pHWP1GFP3 (pGFP1) served as a negative control.

Stable integration of the HWP1 promoter:GFP construct at the genomic enolase locus was achieved by linearization of the construct at the ClaI site in the enolase coding region followed by transformation of CAI4 with 10 μg of DNA. Four transformants were identified; two were stable (GFP1/C2 and GFP1/C3), retaining the plasmid in the absence of selection.

Transformants were first screened by Southern blot analysis to verify integration at the enolase locus. Southern blot experiments were also used to determine the copy number of pHWP1GFP3 DNA inserted into the genome, since it is possible that more than one copy of DNA could become inserted. The copy number of inserted plasmids were determined by comparing hybridization intensities of DNA from transformants with a standard curve of GFP DNA to identify transformants with a single copy of GFP DNA. This procedure was applied in all the experiments to ensure that strains with single integrations were compared for GFP levels.

Southern blot analysis of genomic DNA digested with AvaI confirmed that both the GFP1/C2 and GFP1/C3 transformants had integrated GFP into the ENO locus of C. albicans. The integrated construct produced a 7.8 kb AvaI band that hybridized with both enolase and GFP probes. The undisrupted enolase locus produced a 6.4 kb band. GFP sequences were not found in the parental strain, CAI4.

Several growth conditions were employed to show that the HWP1 promoter:GFP construct was developmentally regulated. GFP1/C2 and GFP1/C3 transformants were grown to stationary phase in YNB (yeast nitrogen base with 50 mM glucose) for 48 hours at room temperature before placement in prewarmed (37° C.) M199, fresh YNB at 27° C., or in the four Lee's media conditions (pH 4.5 and 6.8 at 27° C. and 37° C.) with gentle shaking at the appropriate temperature for 3 hours. The cells were examined for the production of GFP in germ tubes incubated in M199 and Lee's pH 6.8 at 37° C. GFP fluorescence was not localized to germ tubes but was found throughout the cytoplasm of C. albicans cells; GFP does not have a signal sequence or other features that mediate secretion and cell wall expression that are present in HWP1. In addition, the appearance of fluorescence coincided with the induction of germ tube formation. Yeast and pseudohyphae forms did not express GFP. This result showed that the HWP1 flanking sequences present in the plasmid not only permit expression of GFP, but that they also confer hypha-specific regulation to GFP, confirming that the sequences in the plasmid have controlling elements for proper HWP1 expression.

To demonstrate that GFP did not interfere with HWP1 expression, indirect immunofluorescence assays were performed to detect surface HWP1 in the GFP-producing transformants. TEXAS RED® secondary anti-rabbit antibodies were used so that HWP1 could be detected in the presence of GFP. No differences were noted in the appearance of HWP1 on GFP-producing cells compared to wild-type cells and parent strains. The lack of inhibition of HWP1 expression by the GFP construct indicates that artifactual influences on mechanisms regulating HWP1 expression did not occur.

### EXAMPLE 7

### Promoter Elements Regulating HWP1 Expression

Using a 1902 bp fragment of the HWP1 upstream region, fused to GFP, strains with wild-type HWP1 promoter activity were prepared. Fluorescence intensities of such strains at 2.5 hours post-germ tube induction were over 90% of the maximal promoter activity, indicating that sequences responsible for promoter activation were found within this region. Inclusion of additional sequences, up to −2025, did not increase GFP levels. Further, as these strains were generated by integration at the ENO1 locus, native HWP1 locus was not required for promoter activity.

To identify upstream activating sequence (UAS) and upstream regulatory sequence (URS) elements important for GFP expression, sequential deletions of the HWP1 promoter were performed. Promoter deletions were cloned from two genomic C. albicans DNA plasmid clones pBSBglII-1.8 and pBSR1-1.9-16 isolated from an SC5314 genomic library. One plasmid clone contained a BglII fragment of the HWP1 promoter encompassing −1410 to +120, the other contained an EcoRI fragment encompassing −2398 to −425 of the HWP1 promoter.

All polymerase chain reactions (PCR) were performed using PFU TURBO® (STRATAGENE®, La Jolla, Calif.) with dNTPs at 200 μM, oligonucleotides at 0.4 μM, and template DNA at 0.2 ng/μL. External deletions downstream of −1410 were amplified by PCR using oligonucleotides engineered with XhoI and HindIII restriction sites. PCR products were ligated into the reporter plasmid at XhoI and HindIII. External deletions upstream of −1410 were cloned by PCR with XhoI added to the 5′ end and containing the BamHI site (−435) on the 3′ end. PCR products were ligated into pHWP1GFP3 at XhoI and BamHI.

Internal deletions were created by using PCR-ligation-PCR (Ali and Steinkasserer (1995) Biotechniques 18(5):746-50; Abeyrathne and Nazar (2000) Biotechniques 29(6):1172-4, 1176). Internal deletion designated A was constructed by amplifying the two regions −1410 to −1130 and −871 to +60. The DNA was treated with T4 Polynucleotide Kinase (INVITROGEN™ Corp., Carlsbad, Calif.) to add 5′ phosphates, the kinase was heat inactivated, then the two kinase-treated PCR fragments were ligated together with T4 DNA ligase (INVITROGEN™ Corp., Carlsbad, Calif.). The resulting ligation mixture was used as template in a PCR to amplify only the ligations containing the two fragments −1410 to −1130 and −871 to +60 in the correct orientations. The resulting PCR product was digested with XhoI and HindIII and cloned into pHWPIGFP3. Other internal deletions were prepared in similar manner.

Analysis of deletion derivatives (Table 2) showed that the most important activating regions were located between 1 and 2 kb upstream of the transcription start site in that deletion of 840 bp, (−1063) resulted in a 97% reduction in fluorescence. Sequences between −1063 and −555 conferred a basal level of promoter activity.

TABLE 2

| | % of Wild-Type |
|---|---|
| Deletion Construct | Activity |
| −2025 | 89 ± 9.0 |
| −1902 | 90.2 ± 6.0 |
| −1840 | 86.9 ± 4.8 |
| −1782 | 71.6 ± 2.8 |
| −1657 | 77.1 ± 2.0 |
| −1535 | 71.3 ± 5.2 |
| −1410 | 42.0 ± 2.9 |
| −1366 | 73.2 ± 0.3 |
| −1289 | 50.0 ± 3.9 |
| −1242 | 26.8 ± 1.2 |
| −1209 | 11.8 ± 0.9 |
| −1153 | 3.6 ± 0.0 |
| −1063 | 2.4 ± 0.4 |
| −831 | 2.5 ± 0.2 |
| −803 | 2.2 ± 0.3 |
| −555 | 1.4 ± 0.1 |
| −140 | 1.7 ± 0.1 |
| −14 | 1.5 ± 0.2 |

Values represent % of HB-12 wild-type control ± standard deviation.

A bimodal pattern of activation within the 840 bp region between nucleotides −1902 and −1063 was indicated by strains with external deletions. A distal activating region denoted MRR1 (morphogenic response region 1) extended from −1902 to −1410 and accounted for 58% of promoter activity in that deletion of the 5′ 493 bp (−1410) reduced fluorescence to 42% of wild type.

A more proximal region of activation relative to the transcription start site was found spanning nucleotides −1410 to −1042. GFP expression of an internal deletion of this region exhibited 8% of wild-type expression. The importance of this region in activating expression was further demonstrated by fusion of nucleotides −1410 to −1042 to a fragment with only basal promoter activity (external deletion −555; fusion designated E) resulting in a fluorescence intensity that was 67% of wild-type. Attempts to delimit activating sequences within −1042 to −1063 further showed that the entire region was required. GFP expression in strains with a fusion of segment −1288 to −1042 to external deletion −555 (fusion designated K2) was only 12% of wild-type and 17% of construct E. The 3′ boundary of the proximal activating region (−1410 to −1042) was tested in constructs with 3′ ends at −1130 (construct designated A) or −1042 (construct designated B) fused to external deletion −871 having basal promoter activity. Fluorescence intensity attributable to construct A was 22% of fragment B, reflecting the importance of nucleotides between −1130 and −1042. The region between −1410 and −1042 was therefore denoted MRR2. Sequences at the 5′ end of MRR2 repressed as well as activated expression. GFP activity of external deletion −1366 was 73% of wild-type compared to 42% for −1410. This regulatory region within MRR2 was termed MRR2a.

The importance of MRR2 was further illustrated by comparing external deletions −1410 to wild-type in the kinetics of promoter activation. GFP levels were measured over an 8-hour period and reached maximal levels at 7 hours. The magnitude of GFP fluorescence for −1410 was about 50% for each time point. Plots of GFP levels for −1410 and wild-type at 1 hour intervals, divided by the corresponding maximal level at 7 hours, were nearly superimposeable. The results indicate that MRR2 contains sequences essential for activation of the HWP1 promoter and that MRR1 amplifies the effect of MRR2.

To show the importance of the MRR1 and MRR2 in controlling expression of the Hwp1 adhesin, an hwp1/hwp1 null strain was transformed with a promoterless HWP1 gene targeted to the ENO1 locus. Hwp1 was not present as deduced by anti-Hwp1 antibody in an indirect immunofluorescence assay. A control strain, having 1902 bp of HWP1 upstream region fused to the HWP1 coding sequence, produced abundant Hwp1, indicating that upstream sequences harboring MRR1 and MRR2 are required for production of Hwp1 and its associated virulence attributes.

### EXAMPLE 8

### Characterization of DNA Binding Proteins (DNABP) that Regulate the HWP1 Promoter

DNA-binding proteins specific to the cis-activating regions have been identified using electrophoretic mobility shift (EMSA) experiments (Chodosh, (1988) Current Protocols in Molecular Biology, Ausubel et al., eds.) using DNA probes spanning the entire 1902 bp region. Results indicate that all the DNA fragments bind to both yeast and hyphal crude extracts of C. albicans; however, hyphal crude extracts appear to have a higher affinity to upstream region (MRR1 and MRR2) whereas fragments showing basal promoter activity showed strong binding with yeast crude extracts.

To identify the DNABPs involved in regulating HWP1 expression, footprinting experiments coupled with fractionation of proteins in hyphal and yeast extracts are performed, e.g., using DNA affinity chromatography (Myokai, et al. (1999) Proc. Natl. Acad. Sci. USA 96(8):4518-23). Alternatively, a C. albicans cDNA expression library can be screened with the HWP1 upstream sequences identified herein. After the detection and preliminary characterization of proteins that bind to the upstream region of HWP1, the genes for the binding protein of interest are cloned. Having the primary sequence for proteins that bind to the upstream promoter region of HWP1 provides mechanistic information for the morphology-specific gene-expression of HWP1.

To determine the function of the DNABPs in expression of HWP1 as well as in the bud/hyphae transition, gene disruptions are performed. The disruptions are performed using the standard URA-blaster protocol for C. albicans (Fonzi & Irwin (1993) Genetics 134:717-28), which was used to disrupt HWP1. Null mutants of DNABPs that activate HWP1 expression are compared to isogenic strains which express the DNABP in adhesion assays to determine if the loss of the DNABPG affects adhesion. If HWP1 is not expressed, no stabilized adhesion is expected, since HWP1 is the only gene encoding a transglutaminase substrate in C. albicans.

Global regulatory circuits that lead to the production of hypha-specific genes are also identified. Many examples in fungi exist where global regulatory circuits affect multiple genes that are necessary for growth in specific environments. It is likely that C. albicans employs such a regulatory circuit for expressing multiple genes during hypha-specific growth conditions. The DNABPs identified provide a valuable tool for probing the C. albicans genome to identify global regulators.

### EXAMPLE 9

### Mutant Strains of C. albicans Lacking HWP1

Two genomic BamHI fragments containing HWP1 half-clones were isolated from a genomic library constructed in Lambda GEM-12 (PROMEGA®, Madison, Wis.). The BamHI fragments were cloned into PBLUESCRIPT® SK− to generate pGB8 (1.0 kb insert) and pGB23 (3.0 kb insert). An uninterrupted HWP1 gene was created by ligating the 3.0 kb insert of pGB23 into pGB8 to create pGBHWP1. An inactive HWP1 gene was created by replacing HWP1 DNA between the BclI and BglII sites (394 bp) with the 4.0 kb hisG-URA3-hisG cassette of p5921 (Fonzi & Irwin (1993) supra). The new plasmid containing an interrupted HWP1 was named pHWP1URA3.

Both homologues of HWP1 were disrupted (Staab, et al. (1999) supra) using the “URA blaster” method adapted from the protocol developed for use with S. cerevisiae (Alani & Kleckner (1987) Genetics 116:541-5; Fonzi & Irwin (1993) supra). Plasmid HWP1URA3 was digested to completion with HindIII which cuts in the polylinker region and in the HWP1 coding region to release a 5.3 kb fragment harboring the hisG-URA3-hisG disrupted HWP1 gene. Five or 10 μg of digested pHWP1URA3 DNA was used to transform the ura strain CAI4 (Fonzi & Irwin (1993) supra; Postlethwait & Sundstrom (1995) J. Bacteriol. 177:1772-79).

Transformants were screened for the desired recombination events by Southern blot analysis (Postlethwait & Sundstrom (1995) supra). Of the twelve Ura⁺ transformants analyzed, three displayed the desired homologous recombination integration event at the HWP1 locus. One strain, CAH7, was chosen for further manipulations. To remove the URA3 selectable marker, ura auxotrophs of CAH7 were selected on medium containing 5-fluoroorotic acid (Boeke, et al. (1984) Mol. Gen. Genet. 197:345-6; Fonzi & Irwin (1993) supra) prior to the next round of transformations to reestablish the Ura⁺ phenotype. All six Ura⁺ strains examined had the desired loss of URA3 by homologous recombination between the hisG repeats. One strain, CAH7-1, was transformed with the disrupted HWP1 cassette, and Ura⁺ isolates were screened for homologous integration of the transforming DNA at the intact HWP1 locus. Two of nine transformants analyzed harbored the desired disrupted HWP1 loci, and as expected did not have HWP1 on hyphal surfaces as detected by indirect immunofluorescence assay (IFA) (Staab, et al. (1996) supra). An Ura⁻ auxotrophic derivative of CAH7-1A was generated by selection on 5-fluoroorotic medium.

An HWP1⁺ revertant strain was created by using the ura HWP1 strain, CAH7-1A1, as a recipient strain for equal amounts (5 μg each) of HindIII-digested pGBHWP1, and XbaI and XhoI-digested p24enura DNA (Postlethwait & Sundstrom, (1995) supra). Plasmid 24enura harbors an URA3-disrupted C. albicans enolase gene. Ura⁺ transformants were screened for HWP1⁺ phenotype by IFA of germ tubes, and one transformant, CAHR3, was further analyzed by Southern blot analysis. CAHR3 had the desired homologous recombination insertion at the HWP1::hisG locus, and one of the enolase loci was disrupted with URA3. There were no distinguishing phenotypic features between CAHR3 and CAH7-1 (Ura⁺ single disruptant). All of the strains produced germ tubes in liquid and in solid media except when dropped on the surface of agar media. The HWP1⁺ revertant strain (CAHR3) possessed HWP1 on hyphal and not yeast surfaces as detected by IFA. The immunofluorescent results showed that not only was HWP1 gene replacement successful, but that the developmental hypha-specific regulation was also maintained. In other experiments, CAH7-1A was found to lack HWP1 mRNA (Staab, et al. (1999) supra).

### EXAMPLE 10

### HWP1 and Adhesion of Germ Tubes to Human Buccal Epithelial Cells

The isogenic HWP1 strains created above were used to assess the function of HWP1 in adhesion to buccal epithelial cells. HWP1 allowed germ tubes to form stable attachments to BECs that were resistant to treatment with SDS and heat (stabilized adhesion). Stabilized adhesion was abrogated by known inhibitors of transglutaminases, iodoacetamide and monodansylcadaverine. The homozygous HWP1 mutant strain, CAH7-1A, was unable to form stable attachments to BECs whereas the revertant strain and the heterozygous mutant, CAHR3 and CAH7, respectively, were unaffected in the ability to form stable attachments. In biochemical experiments, recombinant HWP1 was shown to be a substrate for mammalian transglutaminase and associate tightly with BECs envelopes after incubations under the adhesion assay conditions (Staab, et al. (1999) supra). The data points to the ability of HWP1 to mediate stable attachments to human tissue through interactions with host transglutaminases. This is further demonstrated by the 78% inhibition of germ tube stabilized adhesion of C. albicans to BECs when the BECs have been preincubated with the N-terminal 148 amino acid residues of Hwp1 (the transglutaminase substrate domain of Hwp1) (Staab, et al. (2004) supra)

### EXAMPLE 11

### HWP1 in Systemic Candidiasis

Mice (6/group) were injected intravenously with wild-type or mutant strains of C. albicans. CAH7-1A, which lacks HWP1, was significantly reduced in the ability to cause lethal candidiasis compared to strains with HWP1.

### EXAMPLE 12

### HWP1 in Gastrointestinal Candidiasis

Beige nude, Epsilon 26 and beige het mice were raised and maintained under germfree conditions. Most mice were used at the age of 4-to-7 weeks; however, a few pups and older animals were also included. Both male and female mice were used. Mice were monoassociated on Day 0 with C. albicans strains CAH7 (HWP1/hwp1), CAH7-1A (hwp1/hwp1), CAHR3 (revertant) or SC5314 (wild-type) by swabbing the mouth with a suspension containing 10⁷ blastoconidia per/mL. Mice were maintained in sterile isolators for up to seven weeks and watched for signs of ill health including lethargy, matted or thinning fur, chills, closed eyes, and emaciation that mandated sacrificing. Thus the terms “ill” and “survival” are synonymous and are used interchangeably in the description below. Fecal pellets were collected to verify that animals were colonized and to verify the identity of strains of C. albicans that the animals had received. Upon sacrificing, quantitative cultures of cecal contents verified the presence of C. albicans in individual animals. Quantitative kidney cultures were performed to determine whether C. albicans had translocated across the gastrointestinal tract.

Gnotobiotic beige nude mice that received C. albicans strains with HWP1 were more prone to failure to thrive than mice that received the C. albicans hwp1 null mutant strain. Only two of seven beige nude mice given the HWP1-containing heterozygous C. albicans strain and one of four mice given the HWP1 revertant remained healthy for six weeks. In contrast, only two of 11 mice that received the hwp1 null mutant strains of C. albicans became ill. The survival difference between groups of mice that received the HWP1 heterozygote strain CAH7 compared to the hwp1 null mutant was statistically significant (P<0.05). The combined groups of mice that received the HWP1 containing heterozygous and revertant, also showed a significant difference in survival from mice receiving the hwp1 null mutant strain. The absence of a significant difference in the survival of mice that received the revertant strain and the hwp1 null mutant (P<0.058) was because one of the mice was sacrificed for “comparison” prior to becoming ill. When this mouse is removed from the analysis, the survival difference between null and revertant is significant.

The survival of Epsilon 26 mice also depended on the presence of HWP1 in C. albicans strains used for monoassociation. All mice that received HWP1-containing heterozygous C. albicans strains and four of five mice given the HWP1 revertant strain became ill. Only one mouse that received the revertant strain remained healthy for seven weeks. In contrast, none of the five mice that received the hwp1 null mutant strains of C. albicans became ill. The survival differences between groups of mice that received the HWP1 heterozygote and revertant strains compared to the hwp1 null mutant were statistically significant (P<0.01).

1. An isolated nucleic acid encoding a Candida albicans HWP1 flanking region, wherein the nucleic acid comprises a nucleotide sequence of SEQ ID NO:1 or SEQ ID NO:2; a nucleic acid that hybridizes to a nucleic acid comprising a nucleotide sequence of SEQ ID NO:1 or SEQ ID NO:2 or its complementary nucleotide sequence under stringent conditions; or a fragment thereof.

2. A vector comprising the isolated nucleic acid of claim 1.

3. A host cell comprising the isolated nucleic acid of claim 2.

4. A method for identifying an agent which modulates the expression of HWP1 comprising contacting a test agent with a host cell expressing a reporter protein, wherein the nucleic acid encoding the reporter is operably linked to an HWP1 nucleic acid of claim 1, and determining whether the test agent modulates the expression of the reporter protein thereby identifying an agent which modulates the expression of HWP1.


SEQUENCE LISTING (25 sequences)

SEQ ID NO: 1 - DNA, 1959 bp, Candida albicans
ggtaattattggaatgaacgtgtttaaacatttgaggaattggtttggagatgagaattggcactaaataggtaattgtttatttttagctgtttcatataatttggcaagtagtttgtactaatgattgtgactattgaaaatgaatttattttttttttcaatttgccttttacaatagtataattttgcgtgtttatatggttatgaacactgtaagctgaatgccataaaaaactcttagctgggtctagtaatgttgtgcatatggttggcagaaatgtcaaaaatgtaagaggatagtatgtcgtgggccgacgcgaatcgtcaaaaaaatccgcctcacaatatttggatcaagatttatgtaaacaaactcccacaaccaatcgtctggcattgaaatataccattttatagtacctatcagtgccatttctgttaatatttggaatattccttgttctttgactttattgctagcatataaagagatctttctttttcatttcccttaaaaccgatcaagaaagaaagtggaaataaagctatgataaatgttgattttgtgtaattcaatcaactaagcacgtttgacagttaaaaagtacgttgttgttgtcctcgtctcgtctaatttctgttgacgaggattaataacaagaaatacaggaaaccctccaaaaaaaaaattttggaccttacacgcacataaattgcggataaacttgccataataaaaactctttgaaacatacgatatgttattcttttcataactggaatatttttgcttttttttaacattatgaacaattgaaaaaaaaaggaaatgaaaaggtaagagttgcctaaccattgaaaataataggctaaggtttttcctgatgcgtttaactaaaaaggaaataacaaaagttattagcgataacctgcgtaaggtgtcaacaaaatatattttgtacgtagcttctatagaaaatatataatctaaatccttaaggaatttcctctatatataataggaaatccctctcacagtgaactgaattatccatctgaattatcagtccactaattccatcaataaaatagattagtgtattgttctcttcagtacaattactaccattatgcaatgctagcttattgttcataattagccatgttgcacaccctaattcgaacattaactgtatccatatttttcttgtccttctttgtttttttctaacaaaaatgttccagaattttttaaaaaatatttgaaaaaacacataacactttgagtatgataatatcaaccattgacttgttttgaaagtaaagaatcaaatttttttctaactcgactaatgcactttacatcaactggatgttatttgcatctactactataagctcaaacaaattatctttcaaaaatgttataattaacaagtcatctataattctttggatccaaaaacaaggaattcggaaattctgacgataaatgtcgactcacaattcattgtaaaaagggagagttttggtaggctcataatcgcttataatgtacctctaaagtaatctaaaacaaacacaacctttctaaaacctataataataaccctaatggctcacaaccgggataagttagttagcccagctgttttttttttgccttatttttatgactacattttgtttcactttttgttgcgactttaataccgtttttgcaacttctctttgtatcacctgtatccgccttttttaacatagcaactcttgtaaagtccctttcttttcccactattttatcattcttgaaatatgtaatcagaatagtttttcaaaaactataaataacggtcaaaataaccggctattttcaatttccattcaacttgttttctcaacaatatcaaacacaacaggaatctcctatagtcactcgcttttagtttcgtcaat

SEQ ID NO: 2 - DNA, 1434 bp, Candida albicans
ttctaataactgatactaagttttgttctttttttgggatttcttttttttctaattttgattgtttttcaattttgggttttcaatattattgacaagagtcattttattgaatatttgttttgtttactacattaaaggtgataggtacttttagtttttaaaaattgttttgttcaaattgtttatctttttcttcttcttctacttgctttgttttctgttttcggttcatagttgatagcttttaataaatacccctttttttttacaatagttagttctaagcttattcagtggtttaattggtagaaaatgtgagctctgtagcttatggtatcttctatagcaatatatttaacttggacatagttcattattctggacgcatgaaggtgcaaagtcaaaaagtgagaatatgcaaagaggtaattagatttctgtcctattaattaacaaaaaatctatatatagactgcaatatttaataccccccaagtttgattctataatccttcgatttctataaagatataaccatgaatcatgagtaaataccaaatagattaatagtagaatctgtatggtcgtgtaaagctgttcattaaaaactattgatttaaatttccccttcctcgtaatctctttttttttgggggggaggatcagccgcaataaaagattgaaaattattaaacaaaacaacaaaacgggaaaccggaaacctgagaaaaagaaagaaagaaggaaaacttttgttaagatattaaattttacgaagcaaatttaaaatccatacttaacaattattgttattacgtacttcaacagggaaagaaaaagttaagattttaaatctctttattcctttctttcttttattattatttacccttaatataaatgtcaaaacaagatccacctccagattatacaaataggacatcagataattataacccagatacaactgataatcataatattcctccacctttcactactcatcctatagaggttcatccaccaccattctcctcttctacttcacctaatatccgtgtgccagcatattttcaaaatcaaactacttcaggatggacaatagtaataaataatcgattttggactgatggattcaggatatttgtttccgaagatgcatctaataagtttgatgccttcaaaaaaagtaaaaatcccgaaataatacaattacaagaacaaggtattggagtaccgttatttaaagctgtcacgtcatatattcccttagcaacaaaatttataacgtttagaagatatgtccctactaatttacatccatttgatattgataaagattattatgattattgtattgttaaacggaaattacacgttggatatgatagttatatatttgaatttactcctgatcgagaattcggatcc

SEQ ID NO: 3 - DNA, 493 bp, Candida albicans
ggtaattattggaatgaacgtgtttaaacatttgaggaattggtttggagatgagaattggcactaaataggtaattgtttatttttagctgtttcatataatttggcaagtagtttgtactaatgattgtgactattgaaaatgaatttattttttttttcaatttgccttttacaatagtataattttgcgtgtttatatggttatgaacactgtaagctgaatgccataaaaaactcttagctgggtctagtaatgttgtgcatatggttggcagaaatgtcaaaaatgtaagaggatagtatgtcgtgggccgacgcgaatcgtcaaaaaaatccgcctcacaatatttggatcaagatttatgtaaacaaactcccacaaccaatcgtctggcattgaaatataccattttatagtacctatcagtgccatttctgttaatatttggaatattccttgttctttgactttattgctagcatataaaga

SEQ ID NO: 4 - DNA, 367 bp, Candida albicans
atctttctttttcatttcccttaaaaccgatcaagaaagaaagtggaaataaagctatgataaatgttgattttgtgtaattcaatcaactaagcacgtttgacagttaaaaagtacgttgttgttgtcctcgtctcgtctaatttctgttgacgaggattaataacaagaaatacaggaaaccctccaaaaaaaaaattttggaccttacacgcacataaattgcggataaacttgccataataaaaactctttgaaacatacgatatgttattcttttcataactggaatatttttgcttttttttaacattatgaacaattgaaaaaaaaaggaaatgaaaaggtaagagttgcctaaccattg

SEQ ID NO: 5 - DNA, 10 bp, Candida albicans
ctgaattatc

SEQ ID NO: 6 - DNA, 7 bp, Candida albicans
tataaat

SEQ ID NO: 7 - DNA, 8 bp, Candida albicans
gcacgtta

SEQ ID NO: 8 - DNA, 6 bp, Candida albicans
atgata

SEQ ID NO: 9 - DNA, 6 bp, Candida albicans
cggata

SEQ ID NO: 10 - DNA, 6 bp, Candida albicans
acgata

SEQ ID NO: 11 - DNA, 6 bp, Candida albicans
gcgata

SEQ ID NO: 12 - DNA, 6 bp, Candida albicans
tatcca

SEQ ID NO: 13 - DNA, 6 bp, Candida albicans
tatcag

SEQ ID NO: 14 - DNA, 6 bp, Candida albicans
tatcca

SEQ ID NO: 15 - DNA, 6 bp, Candida albicans
tatcaa

SEQ ID NO: 16 - DNA, 6 bp, Candida albicans
tatctt

SEQ ID NO: 17 - DNA, 6 bp, Candida albicans
gggata

SEQ ID NO: 18 - DNA, 6 bp, Candida albicans
tatcac

SEQ ID NO: 19 - DNA, 6 bp, Candida albicans
tatccg

SEQ ID NO: 20 - DNA, 6 bp, Candida albicans
tatcat

SEQ ID NO: 21 - DNA, 12 bp, Candida albicans
aagcacgtttga

SEQ ID NO: 22 - DNA, 12 bp, Candida albicans
ttgcacgttagc

SEQ ID NO: 23 - protein, 50 aa, Candida albicans
CTNCGTKTTPLWRRNPQGQPLCNACGLFLKLHGVVRPLSLKTDVIKKRQR

SEQ ID NO: 24 - protein, 50 aa, Candida albicans
CTNCFTQTTPLWRRNPDGQPLCNACGLFLKLHGVVRPLSLKTDVIKKRNR

SEQ ID NO: 25 - RNA, 6 nt, Candida albicans
aauaaa